(19) United States
(12) Patent Application Publication
(10) Pub. No.: US 2008/0193422 A1
(43) Pub. Date: Aug. 14, 2008

(54) Method for Activating Cd4 Cell

(75) Inventors: Yong-Keun Park, Seoul (KR); Won-Suck Yoon, Seoul (KR)

Correspondence Address: THE RAFFERTY PATENT LAW FIRM, 5641 BURKE CENTRE PKWY, SUITE 100, BURKE, VA, 22015-2259, US

(73) Assignee: KOREA UNIVERSITY INDUSTRY AND ACADEMY COOPERATION, Seoul (KR)

(21) Appl. No.: 11/912,148

(22) PCT Filed: Jan. 20, 2006
(86) PCT No.: PCT/KR2006/000229

§ 371 Date: Oct. 21, 2007

(30) Foreign Application Priority Data
Apr. 21, 2005 (KR) 10-2005-0033189

(51) Int. Cl. A61K 35/14; C12N 5/00; A61P 31/04

(52) U.S. Cl. 424/93.7; 435/375

(57) ABSTRACT

A method for activating a CD4 T cell includes (1) separating CD4 T cells; and (2) cultivating them in vitro with a culture broth containing cytokines such as GM-CSF, IFN-γ, TNF-α, lectin and IL-4. Also, a therapeutic composition for preventing or treating infectious diseases of bacteria includes CD4 T cells activated by the above-noted method.

### CROSS-REFERENCES TO RELATED APPLICATIONS

This is a US National Stage of International Application PCT/KR2006/000229, filed with the Republic of Korea Receiving Office on Jan. 20, 2006. The benefit of priority is further claimed to Republic of Korea patent application 2005-0033189, filed Apr. 21, 2005.

### TECHNICAL FIELD

The present method relates to activating a CD4 T cell, and may include the following steps: separating CD4 T cells, and cultivating them in vitro with a culture broth containing cytokines such as GM-CSF, IFN-γ, TNF-α, lectin and IL-4. In addition, a therapeutic composition is provided for preventing or treating infectious bacterial diseases, which includes the CD4 T cell activated by the present method.

### BACKGROUND

Salmonella typhimurium brings about diarrhea and enteritis in human and is a causative agent of food poisoning often found in the intestinal remnant of cattle, including pigs. When eating dirty water or food contaminated with animal feces, food poisoning may be caused, even in human beings. This infection of Salmonella sp. has typically been treated using a penicillin series antibiotic.

In the case of recombinant Salmonella bacteria intentionally made for biological terror and antibiotic-resistant Salmonella recently found in Germany, however, general antibiotics are no longer effective to treat these infections. In practice, suitable replacements for such therapeutic methods are lacking, even when concerning damages.

In the research field of drugs, immune cells, etc., are being investigated to develop a therapeutic agent. For this therapeutic method, the immune cells are collected from a particular subject and activated before being administered to a patient directly. Presently, immune cells activated by APC cell and specific antibody, etc., have been chosen for therapeutic use.

### SUMMARY

In view of the above-mentioned, as well as other problems, the present inventors have found that a CD4 T cell is activated synergistically by adding a culture broth containing GM-CSF, IFN-γ, TNF-α, lectin and IL-4, and further, the resulting CD4 T cell can treat infectious bacterial diseases and prevent their re-infections effectively.

An object of the present invention is to provide a method for in vitro activation of a CD4 T cell that may include separating CD4 T cells from a biological specimen of subject, and cultivating them with a culture broth containing cytokines including GM-CSF, IFN-γ, TNF-α, lectin and IL-4.

Another object of the present invention is to provide a therapeutic composition for preventing or treating infectious bacterial diseases that includes the CD4 T cell activated by the above-noted method.

### BRIEF DESCRIPTION OF THE DRAWINGS

The above and other objects, features and other advantages of the present invention will be more clearly understood from the following detailed description taken in conjunction with the accompanying drawings, in which:

FIG. 1 depicts the process for activating CD4 T cell after separating it from a subject schematically;

FIG. 2 depicts the comparison of cell efficacies on mice infected by Salmonella bacteria;

FIG. 3 depicts the test of vaccine efficacy on Salmonella infection in mice survived after administering the activated CD4 T cell; and

FIG. 4 depicts the antigen-antibody reaction against Salmonella bacteria in sera collected from mice survived after administering the activated CD4 T cell.

### DETAILED DESCRIPTION

In one embodiment of the present invention, a method for in vitro activation of a CD4 T cell is provided, which includes separating CD4 T cells from a biological specimen of subject, and cultivating the CD4 T cells with a culture medium containing cytokines including GM-CSF, IFN-γ, TNF-α, lectin and IL-4.

The T cell denotes a T cell population that participates in an immune reaction and expresses a specific marker on the cell surface. As one example in accordance with the present invention, only the T cell having a CD4 marker is selected among the T cell population. Preferably, the T cell having the CD4 marker may include a helper T cell (TH cell) that activates both cellular and humoral immune reactions, and/or a delayed type hypersensitivity T cell (TD cell).

In the present description, “subject” refers to a donor providing a mononuclear cell such as a T cell, and may include any organism having a vascular system and hematopoietic cells. Preferably, the subject includes a vertebrate selected from among cows, horses, sheep, pigs, goat, camels, antelopes, dogs, mice and the like.

The T cell—i.e., the CD4 T cell—can be isolated from various biological samples including mononuclear cells. Preferably, the T cell can be purified from among blood, plasma, lymph node, spleen, thymus, bone marrow and the like.

The process for isolating the T cell is not limited to any particular technique. Preferably, the T cell can be isolated by depending upon cell density, affinity of antibody against cell surface epitope, cell size, and/or degree of fluorescent emission. For example, the T cell may be isolated by conducting a density gradient centrifugation using albumin, dextran, Ficoll, metrizamid, Percoll and the like; (MACS) etc. using an antibody; a centrifugal elutriation, etc., depending upon cell size; and/or a FACS using fluorescence.

In accordance with the present invention, the T cell may be isolated by performing a magnetic activated cell sorter using an anti-CD4 T cell antibody in order to purify a CD4 T cell from a mononuclear cell exclusively.

The CD4 T cell isolated above is cultivated by a conventional method and activated in vitro. Preferably, the T cell is cultivated using a culture medium containing nutrients essential for cell growth and survival. Also preferably, the culture medium is comprised of various sources of carbon, nitrogen and trace elements; more preferably, a culture media containing serum; and most preferably, commercial media such as DMEM and RPMI.

In the present description, “activation of T cell” refers to stimulating a T cell to participate in an immune reaction. The T cell can be activated by various signals. In view of the objects of the present invention, the activation of T cell denotes an activation of a CD4 T cell.

A CD4 T cell may be activated synergistically by adding a culture mixture containing GM-CSF, IFN-γ, TNF-α, lectin and IL-4. As an example, experimental mice were observed to have a 90% survival rate when injected with CD4 T cells activated by cytokines according to the above-mentioned process, despite a 100% death rate otherwise to be expected upon infection by Salmonella typhimurium. In contrast, experimental mice were observed to have only a 20% survival rate when injected with CD8 T cells. As a consequence, the CD4 T cell is very effective to treat infectious diseases caused by Salmonella bacteria.

In addition, the experimental mice that survived by injecting the CD4 T cells were observed to avoid re-infections by Salmonella bacteria. As a consequence, the CD4 T cell has an outstanding efficacy as a vaccine.

In the method for activating a CD4 T cell in vitro, the concentration of cytokines included in the culture broth are adjusted in the ranges of between 0.05 and 0.2 μg/ml of GM-CSF, between 0.5 and 2 μg/ml of IFN-γ, between 0.05 and 0.2 μg/ml of TNF-α, between 40 and 60 μg/ml of lectin and between 0.05 and 0.2 μg/ml of IL-4.

The cytokine is a wild type of GM-CSF, IFN-γ, TNF-α, lectin or IL-4 derived from various subjects, but can be a fragment or variant of GM-CSF, IFN-γ, TNF-α, lectin or IL-4, if it retains the biological activity of the wild type. The culture broth containing the cytokines can include additional ingredients additionally. Preferably, the additional ingredient may include IL-5, IL-10, IL-13 and the like.

Within an effective dose, the process of adding the cytokine mixture into a culture broth, the timing, and the number of addition are not particularly limited in order to activate the CD4 T cell. The cytokine mixture can be added while the CD4 T cell is cultivated or, alternatively, several times in some interval after being cultivated. In addition, within an effective dose, the cytokine mixture can be administered once or multiple times and the timing of adding each cytokine can be controlled properly. Preferably, all the cytokines can be treated once while the cell is cultivated. Preferably, the CD4 T cell is further cultivated for 1 to 4 days even after the cytokines are added.

In accordance with the present invention, a simple and effective method for stimulating CD4 T cell activity is provided and may be an important factor determining the efficiency of cell therapy without toxicity. The resulting CD4 T cell activated by the above-mentioned method can be applied to treat various kinds of cancers, bacterial infections and immune diseases, as a biological medicine for multiple uses.

In another embodiment, a therapeutic composition for preventing or treating infectious diseases of bacteria is provided that includes a CD4 T cell activated by the above-mentioned method.

In still another embodiment, a method is provided for preventing or treating infectious diseases of bacteria by administering a therapeutically effective amount of CD4 T cells activated by the above-mentioned method to a patient.

In the present description, “prevention” designates any activity that inhibits a bacterial infection or delays an infection by administering the CD4 T cell, activated as noted above, to a patient. Also, “treatment” means any activity that improves or advantageously modifies a symptom of bacterial infection by administering the CD4 T cell (activated as noted above) to a patient. “Patient” denotes a human being or an animal, such as a cow, horse, sheep, pig, goat, camel, antelope, dog and the like, that can undergo improvement with respect to an infectious bacterial disease by administering the CD4 T cell activated as noted above. The infectious diseases of bacteria can be prevented or treated effectively by administering the CD4 T cell activated as noted above in a cytokine mixture comprised of GM-CSF, IFN-γ, TNF-α, lectin and/or IL-4 to a patient.

In the method of the present invention, the infectious disease that can be treated by administering the CD4 T cell activated as noted above is not limited to any particular disease, but preferably may include an infectious disease caused by Salmonella bacteria. Preferably, the Salmonella bacteria can be Salmonella arizonae, Salmonella choleraesuis, Salmonella enteritidis, Salmonella typhi, Salmonella typhimurium and the like. More preferably, the infectious disease is caused by Salmonella typhimurium.

The CD4 T cell can be administered through any pathway, if possible to reach a target tissue. For example, the cell can be administered parenterally, such as by intra-peritoneal injection, intravenous injection, intramuscular injection, subcutaneous injection or intra-dermal injection, but it is not necessarily limited thereto. The cell composition of the present invention can be administered using any apparatus suitable to transport active ingredients to a target cell. The cell composition can be additionally comprised of conventional pharmaceutical carriers suitable for cell therapy, such as physiological saline solution.

The CD4 T cell of the present invention is preferably administered in a therapeutically effective amount. In the present description, “therapeutically effective amount” designates an amount sufficient to treat a disease in a reasonable ratio of benefit/risk suitable for medical therapy. The dosage to be ingested may vary, depending on factors such as severity of disease, age, sex, time of administration, method of administration, ratio of discharge, period of treatment, other drugs and medical factors already known in the art. It is important to administer a minimal amount effective in a maximal extent without any adverse action, considering all the factors. The dosage may be determined by those skilled in the art. As a general guide, it is expected that an adult patient may ingest at one time about 1 mg to 1,000 mg of the CD4 T cells according to the present invention. For individual patients having particular body weights and lifestyles, a proper dosage may be readily determined by starting out with the general dosage level set forth above and adjusting the dosage as necessary to alleviate the disease.

Practical and presently preferred embodiments of the present invention are illustrated as shown in the following Examples. However, it will be appreciated that those skilled in the art, on consideration of this disclosure, may make modifications and improvements within the spirit and scope of the present invention.

### EXAMPLE 1

### Preparation of CD4 T Cell Alleviating Salmonella Infection

Spleens of mice (BALB/c, SLC Japan) were obtained and sonicated with a cell grinder. The resulting cells suspended in RPMI medium were centrifuged at 1,500 rpm, and then 10 ml of RBC lysing buffer (Sigma, USA) was added to react for 10 minutes at room temperature. After that, the result was centrifuged at 1,500 rpm to remove erythrocytes by exchanging 10 ml of RPMI media three times. The mononuclear immune cells obtained above were separated by performing the MACS method using anti-CD4 T cell antibodies to obtain CD4 T cells. The resulting CD4 T cells were cultivated for 48 hours with RPMI media containing 10% FBS and cytokines.

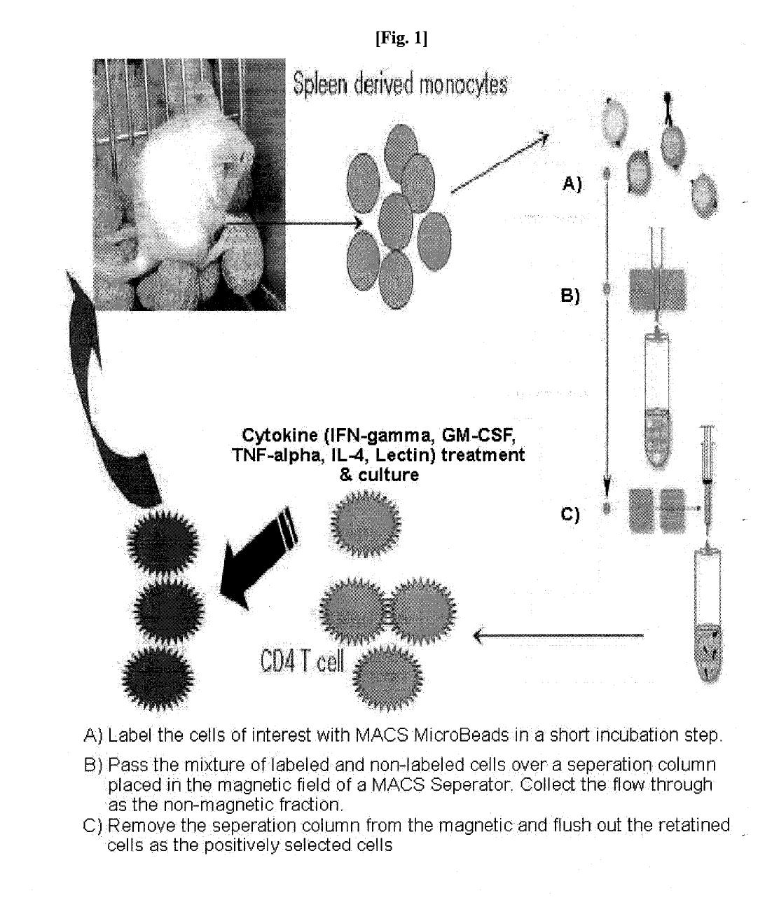

The cytokines added were adjusted in their amounts to GM-CSF 0.1 μg, IFN-γ 1 μg, TNF-α 0.1 μg, lectin 50 μg, and IL-4 0.1 μg, respectively. The cultured cells were collected and then suspended with PBS buffer before being applied for cell therapeutics. FIG. 1 schematically depicts the process for activating the CD4 T cell after separating it from a subject.

### EXAMPLE 2

### Examination of Efficacy of Cell Therapeutic after Salmonella Infection in Mouse Model

In order to induce a bacterial infection, 1×10⁴ Salmonella typhimurium bacteria (Korea University) were orally administered to mice. By the same procedure described in Example 1, CD4 T cells were activated and suspended with PBS buffer to reach 1×10⁶ of cell concentration. Then, the resulting cells were injected intravenously into experimental mice infected by Salmonella bacteria.

The survival ratios of mice were measured and compared—in the control group, injecting only PBS buffer; and the experimental group, injecting a macrophage cell line or the CD8 T cell (See Table 1).

TABLE 1

| | control | macrophage | CD8 T cell | CD4 T cell |
|---|---|---|---|---|
| | (surviving | (surviving | (surviving | (surviving |
| day | mice/all mice) | mice/all mice) | mice/all mice) | mice/all mice) |
| 2 | 10/10 | 10/10 | 10/10 | 10/10 |
| 4 | 8/10 | 10/10 | 10/10 | 10/10 |
| 8 | 2/10 | 10/10 | 10/10 | 10/10 |
| 10 | 0/10 | 6/10 | 8/10 | 10/10 |
| 12 | 0/10 | 0/10 | 7/10 | 9/10 |
| 14 | 0/10 | 0/10 | 2/10 | 9/10 |
| 16 | 0/10 | 0/10 | 2/10 | 9/10 |

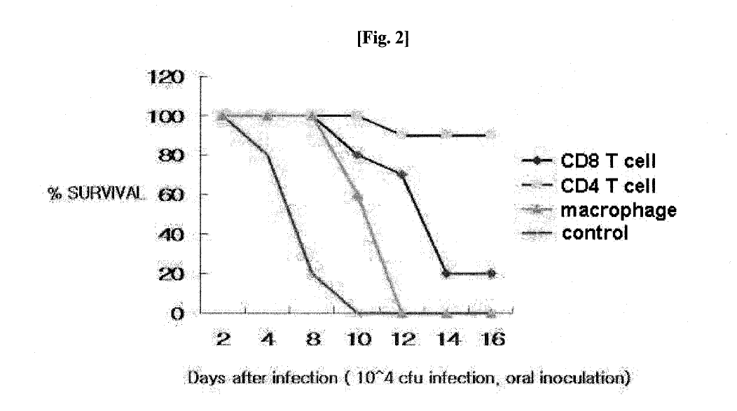

FIG. 2 depicts the comparison of cell efficacies on mice infected by Salmonella bacteria. As a result, it is observed that the CD4 T cell activated as noted above increases a survival ratio of the mice infected by Salmonella bacteria after it is administered.

### EXAMPLE 3

### Examination of Vaccine Efficacy of Cell Therapeutic on Infectious Disease of Salmonella in Mouse Model

Experimental mice were infected by Salmonella bacteria and then treated with the CD4 T cells activated by the same procedure described in Example 1. Nine mice of the surviving group above were again administered 1×10⁴ Salmonella bacteria. In contrast, nine mice of the control group, which had never been infected by Salmonella bacteria and which had been injected with only PBS buffer, were administered with Salmonella bacteria as described above. Survival ratios were compared in the two groups to evaluate the efficacy of the vaccine after administering the CD4 T cells.

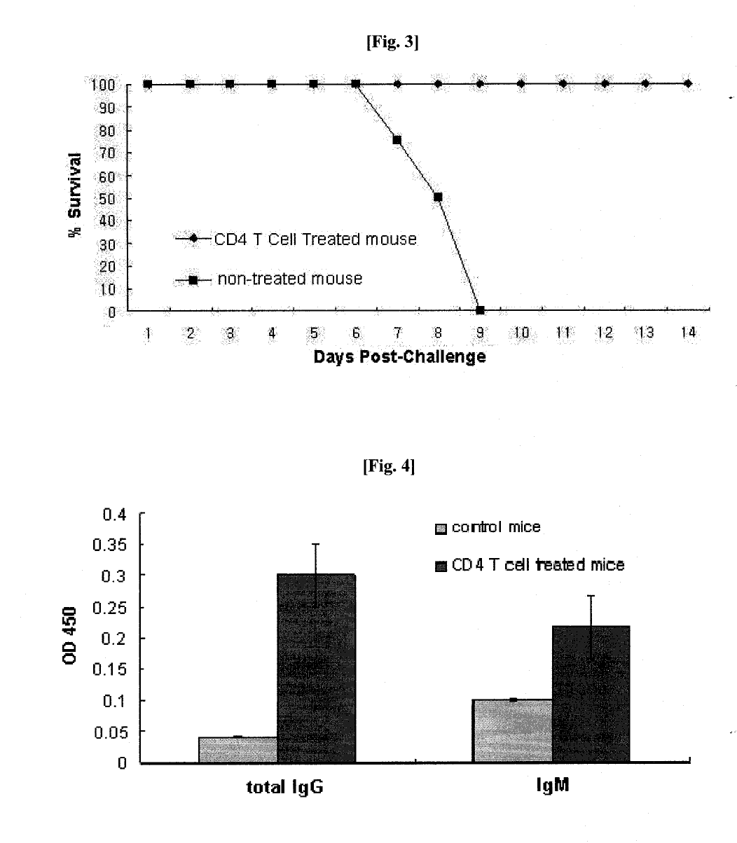

FIG. 3 depicts the test of vaccine efficacy on Salmonella infection in mice that survived after administering the activated CD4 T cell. As a consequence, it is confirmed that the experimental mice that survived as noted above were completely prevented from experiencing re-infection by Salmonella bacteria by injecting the CD4 T cell of the present invention.

### EXAMPLE 4

### Examination of Antigen-Antibody Reaction against Salmonella Strains

In order to investigate the vaccine efficacy of the cell therapy, the experimental mice were treated with the cells and if they survived, were operated upon to collect blood from their eyeballs. The blood of the mice was centrifuged at 3,000 rpm in order to obtain serum. The resulting serum was examined to measure the degree of antibody reaction specific for an antigen of Salmonella bacteria by using the ELISA method.

FIG. 4 depicts the antigen-antibody reaction against Salmonella bacteria in serum collected from mice that survived after administering the activated CD4 T cell. As illustrated in FIG. 4, it is observed that the mice treated with the CD4 T cells indicates that the antibody reaction (total IgG) is approximately 10-fold sensitive to Salmonella bacteria, compared to the normal mice without any treatment. Further, it is observed that the mice treated with the CD4 T cells have an approximately two-fold IgM value, an immunity index of viscous membrane, compared to the normal mice. Therefore, it is confirmed that the experimental mice treated with the cell therapy may stimulate an antibody reaction specific for Salmonella strains.

As illustrated and confirmed above, the CD4 T cell activated by the method of the present invention may treat and prevent infectious bacterial diseases including Salmonella sp., etc., effectively.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for in vitro activating a CD4 T cell that comprises steps: (1) separating CD4 T cells from a biological specimen of subject; and (2) cultivating the CD4 T cells with a culture medium containing cytokines including GM-CSF, IFN-γ, TNF-α, lectin and IL-4.

2. The method for in vitro activating a CD4 T cell according to claim 1, wherein the biological specimen is one selected from a group consisting of blood, plasma, lymph node, spleen, thymus and bone marrow.

3. The method for in vitro activating a CD4 T cell according to claim 1, wherein the concentration of cytokines are adjusted in the ranges of 0.05 to 0.2 μg/ml of GM-CSF, 0.5 to 2 μg/ml of IFN-γ, 0.05 to 0.2 μg/ml of TNF-α, 40 to 60 μg/ml of lectin and 0.05 to 0.2 μg/ml of IL-4.

4. The method for in vitro activating a CD4 T cell according to claim 1, wherein the CD4 T cells are cultivated for 1 to 4 days in Step (2).

5. A therapeutic composition for preventing or treating infectious diseases of bacteria which comprises the CD4 T cells activated by the method of claim 1.

6. The therapeutic composition according to claim 5, wherein the bacteria are Salmonella spp.

7. The therapeutic composition according to claim 6, wherein the bacteria are Salmonella typhimurium.
